(19) United States
(12) Patent Application Publication
(10) Pub. No.: US 2005/0008764 A1
(43) Pub. Date: Jan. 13, 2005

(54) Method and apparatus for moistening clean room wipers

(75) Inventors: Thomas Edward Priebe, Shakopee, MN (US); Valentin Atanssov Kozov, Plymouth, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Seagate Technology LLC, Scotts Valley, CA (US)

(21) Appl. No.: 10/616,786

(22) Filed: Jul. 10, 2003

(51) Int. Cl. B05D 1/00; B05C 5/00

(52) U.S. Cl. 427/8; 427/430.1; 118/300; 118/688

(57) ABSTRACT

A device for uniform moistening of cleanroom wipers. The device includes a wetting chamber where nozzles are directed toward wipers placed on a rack. A liquid supply and a pump with a user interface are connected to the wetting chamber. A pump control in conjunction with a feedback device regulates the amount of liquid dispensed to the wipers to meet a target saturation level as inputted into the user interface. The saturation level of the wipers are monitored by one of several parameters which include time, mass, conductivity or pressure.

### CROSS-REFERENCE TO RELATED APPLICATION(S)

None.

### BACKGROUND OF THE INVENTION

This invention relates to a device which uniformly moistens a material, such as a cleanroom wiper, for use in a cleanroom.

In laboratory work and production of precision parts, it is necessary to have a room that is maintained virtually free of contaminants such as dust and or bacteria. This room is know as a cleanroom or a white room. The contamination in a cleanroom must be kept to a minimum as even low levels of particles or bacteria can damage the production of a product.

Cleanroom wipers are available in many forms. One of the most common forms is a stack of individual wipers pre-moistened with a liquid solution, usually isopropyl alcohol and water, packaged in a container. However, with time the saturation level of the wipers varies from sheet to sheet as gravity causes the liquid to move to the bottom of the wiper container. Therefore, a pool of liquid may form at the bottom of the container leaving the wipers on top drier, while the wipers on bottom are wetter.

This non-uniformity of saturation can create problems regardless of the environment in which the wipers are used. Two examples are the cleanroom and a biological laboratory. In a cleanroom, wipers with a low level of saturation are desired because they clean better without leaving any residual liquid. When pressed against the surface to be cleaned, the liquid from a wiper with a lighter load will come out of the wiper and interact with the particles to loosen them from the surface. When the pressure is removed, the liquid goes back into the wiper and does not stay on the surface. The opposite is true in a biological laboratory where wipers with a high level of saturation are desirable. Highly saturated wipers will leave an amount of residual cleaning solution, such as a germicide, behind. This allows the cleaning solution to interact with the surface even after the wiper has been removed.

Attempts to address the non-uniformity of saturation between multiple wipers have included placing a barrier between wipers to prevent water from migrating to the bottom of the container. The use of the barriers are still problematic as maintaining these barriers for a large number of wipers in a container would be costly. Also, the barriers themselves can add contamination to the wipers.

Spraying individual wipers by hand has also been used as a method of trying to achieve a uniform saturation level within the wiper itself and between wipers at the point of use. Wetting wipers by hand proves less than ideal as the saturation level across the wiper is very difficult to control when a spray or squirt bottle is used. Additionally, hand spraying the wiper does not allow for uniform wetting of wipers across multiple layers.

Another problem with the moistening of wipers is the saturation level of within the wipers itself. One attempt to address the non-uniformity of saturation in the wipers itself involves wetting cleanroom wipers at the point of use by breaking a liquid container inside the wiper container. However, this does not ensure uniform wetting of wipers as the majority of the solution will be absorbed by the wiper in the area in which the liquid packet was broken.

Yet another problem with pre-moistened wipers is the possibility of the degradation of the wiper material. As the wiper continues to sit in the solution, the fibers of the wiper material may decompose and create particulate. After a certain point, the longer the material is saturated and the greater the degradation of the fiber, the dirtier the material will be. This is especially true if the wiper is made from polyester and has been soaked in an isopropyl alcohol solution. Cleaning with material that has degraded is detrimental to the cleanroom environment as the material will shed and leave residual particle matter. The greater the degradation, the more residual particle matter that is left behind.

Therefore, there is a need for a product that provides a uniform saturation level within the wipers itself and through a stack at the point of use.

### BRIEF SUMMARY OF THE INVENTION

The present invention includes a wiper moistening machine where dry, cleanroom wipers are saturated for the first time at the point of use. The machine provides for moistening of a single wiper or a small stack of wipers. Wipers are placed on a rack inside an enclosed wetting chamber that is designed to contain sprayed liquids. An applicator applies liquid to the wiper. A control regulates the application of the liquid to the wiper. The control works in conjunction with a feedback system to achieve a specified target saturation level. The saturation level of the wiper can be controlled by any one of several parameters, such as time, mass, conductivity or pressure.

In preferred embodiments, the bottom of the chamber has a drainage and collection system to collect any liquid spray that misses the wiper. The machine preferably is portable so that it can be moved easily inside the cleanroom.

### BRIEF DESCRIPTION OF THE DRAWINGS

FIG. 1 is a block diagram of a moistening device for uniformly and repeatably wetting wipers.

FIG. 2 is a perspective view of a wetting chamber of the moistening device with the door open.

FIG. 3 is a diagrammatic view of a cross section of the interior of the wetting chamber in use.

FIGS. 4A and 4B are pattern diagrams of alternative spray nozzle configurations.

FIGS. 5A and 5B are pattern diagrams of additional alternative spray nozzle configurations.

FIG. 6 is a diagrammatic view of a cross section of the interior of an alternative embodiment of the wetting chamber.

### DETAILED DESCRIPTION

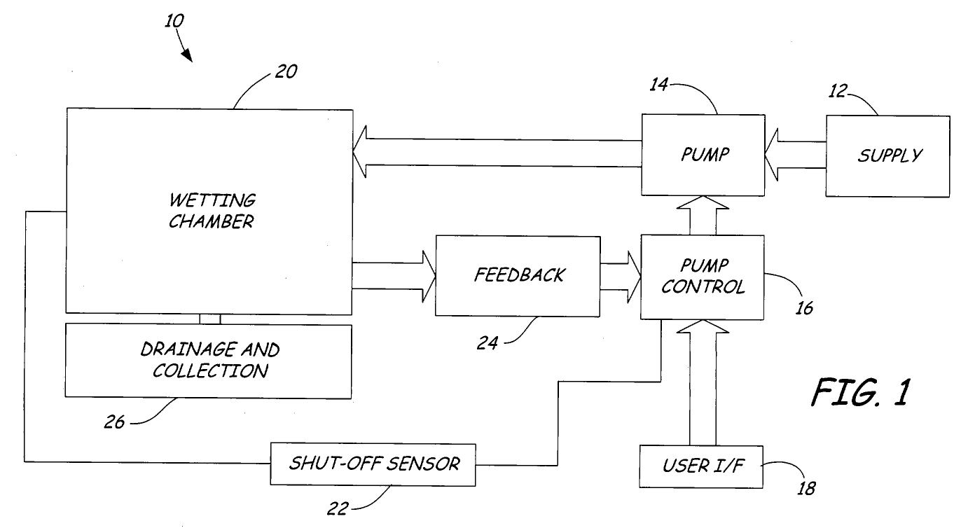

The present invention allows for uniformly and repeatably moistening a single cleanroom wiper or a small stack of wipers at the point of use. FIG. 1 is a block diagram of moistening device 10 which allows a user to moisten material for use in a cleanroom, such as a cleanroom wiper or stacks of wipers with each use. Other materials may also be used with the device such as swabs, fabrics, cloths and paper products. Additionally, device 10 may also be used in other settings including, but not limited to laboratories, biomedical areas, surgical areas, hospitals and manufacturing facilities. Moistening device 10 includes liquid supply 12, pump 14, pump control 16, user interface 18, wetting chamber 20, shut-off sensor 22, feedback device 24 and drainage and collection system 26.

Liquid supply 12 is connected to pump 14. Any suitable pump can be used as pump 14 in device 10. One example of a suitable pump is a syringe pump. Attached to pump 14 is pump control 16, which receives control settings from user interface 18. User interface 18 allows a user to set device 10 to a target saturation level.

Pump 14 is also connected to wetting chamber 20 wherein wipers are moistened by a liquid spray. Wetting chamber 20 has an access means (such as a door, hatch, drawer or the like) by which wipers (not shown) can be placed inside wetting chamber 20. Attached to wetting chamber 20 is shut-off sensor 22 that causes pump 14 to discontinue the flow of liquid to wetting chamber 20 when the access means is open. Also connected to wetting chamber 20 is feedback device 24 which provides a feedback signal from wetting chamber 20 to pump control 16.

Wipers are wetted in moistening device 10 by placing wipers in wetting chamber 20. A target saturation level is inputted into user interface 18.

Pump 14 then, under the direction of the pump control 16, draws liquid from the liquid supply 12 to wetting chamber 20 to moisten the wipers. Feedback device 24 provides a feedback signal regarding a parameter related to the saturation level of the wiper to pump control 16.

Using the feedback signal from feedback device 22, pump control 16 regulates the disbursement of the liquid solution to the wiper via a specified parameter. Three exemplary parameters are time, mass and conductivity. A first suitable control parameter for the measurement of the saturation level of the wiper is time. A specified amount of time is inputted into user interface 18 of control pump 16. When the specified duration of time has elapsed, pump control 16 directs pump 14 to discontinue the disbursement of liquid to the wiper. By knowing the desired volume to be applied to the wiper and the flowrate of the liquid being pumped into wetting chamber 20, the time necessary for the application of a certain amount of liquid to the wipers can be determined. With a set flowrate of liquid, a certain volume of liquid would be delivered to wetting chamber 20 and time/volume would correlate to the saturation level of the wiper.

A second suitable control parameter is the mass of the wiper as liquid is applied to the wiper. A specified mass is inputted through user interface 18 to pump control 16. As the liquid is added to the wiper, feedback device 24 senses the mass of the wiper and volume of liquid. Feedback device 24 then relays the sensed mass information back to pump control 16. When the sensed mass reaches the target level as inputted by user interface 18, pump control 16 directs pump 14 to discontinue the disbursement of liquid to the wiper. The increased mass of the wiper correlates with the volume of liquid that was added to the wiper, therefore, the mass necessary for a specified saturation level can be determined. Feedback device 24 (or pump control 16) can be adjusted to compensate for any liquid mass that might be on the rack itself rather than the wiper.

A third suitable control parameter is the electrical conductivity of the wiper as liquid is added to the wiper. The conductivity of the wiper would correlate to the level of saturation in the wiper, therefore, a specified target saturation level can be determined by the conductivity of the wiper. A specified conductivity level is inputted through user interface 18 to pump control 16. As the liquid is added to the wiper, feedback device 24 monitors the electrical conductivity of the wiper. Feedback device 24 then relays the information back to the pump control 16. When the conductivity level reaches the target level as inputted through user interface 18, pump control 16 directs pump 14 to discontinue the disbursement of liquid to the wiper.

Moistening device 10 also has a feature for the drainage and collection of any excess liquid. Connected to wetting chamber 20 is a drainage and collection system 26. Any excess liquid not absorbed by the wiper is drained from wetting chamber 20 into a collection receptacle. The drainage and collection system 26 allows for ease of collection and disposal of any excess liquid, especially liquid that might have any adverse environmental effects.

Moistening device 10 allows for uniform and repeatable moistening of a single wiper or a small stack of wipers. The same saturation levels can be obtained every time a wiper is placed in the wetting chamber. This allows a user the comfort of knowing that a wiper will have the same saturation level as long as the same specified target is inputted into user interface 18.

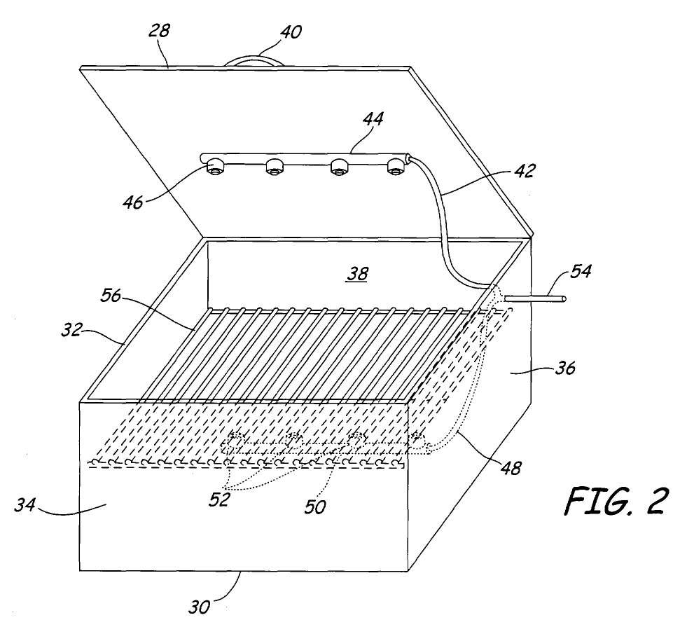

FIG. 2 is a perspective view of the wetting chamber 20, where wipers are placed for moistening, with the top open. Wetting chamber 20 includes a container with a top wall 28, bottom wall 30 and four sidewalls 32, 34, 36, 38. Wetting chamber 20 also includes handle 40, upper tubing 42, upper manifold 44, upper set of spray nozzles 46, lower tubing 48, lower manifold 50, lower set of spray nozzles 52, tubing 54 and rack 56.

Wetting chamber 20 is an enclosed six sided container designed to contain sprayed liquid. Top wall 28 of wetting chamber 20 is attached to rear sidewall 38 of the wetting chamber 20. This attachment can be done using various methods, including, but not limited to hinges, joints and springs. Additionally, top wail can be attached to any of.the sidewalls 32, 34, 26, 38 and is not limited to sidewall 38. Handle 40 is attached to the exterior of top wall 28 to provide access to wetting chamber 20. Any suitable means with sufficient strength to hold the weight of top wall 28 can be used in place of handle 40 such as a knob, latch or cord.

Attached to the interior of top wall 28 is upper manifold 44 wherein a set of upper spray nozzles 46 is affixed to upper manifold 44. Any suitable nozzle may be used in wetting chamber 20. Examples of suitable nozzles include, but are not limited to those with a round spray, square spray and flat spray pattern. Also suitable are full cone, hollow cone and air assisted nozzles. Liquid is dispensed to spray nozzles 46 through upper tubing 42 which is connected to upper manifold 44.

Upper tubing 42 is joined with tubing 54 which is inserted through an opening in sidewall 36 to connect with pump 14. An opening can be created in any of the other sidewalls 32, 34 or 38, or top wall 28 or bottom wall 30 in which tubing 54 can be inserted to connect with pump 14. Tubing 54 is also joined with lower tubing 48 which is connected to lower manifold 50 to dispense liquid to a set of lower spray nozzles 52. Lower spray nozzles 52 are inset into lower manifold 50 which is attached to bottom wall 30. In regards to connectors 42, 48 and 54, any suitable fluid connection material can be used including hoses and pipes.

Centrally located between upper spray nozzles 46 and lower spray nozzles 52 is rack 56. Rack 56 can be comprised of any suitable material sturdy enough to contain a small stack of wipers. Examples of suitable materials include metal, plastic and ceramic. Rack 56 can also be removably or permanently attached to one or more sidewalls 32, 34, 36 and/or 38 or bottom wall 30. Regardless if rack 56 is removable or permanent, there are various suitable methods in which rack 56 can be secured into position in wetting chamber 20 including, but not limited to; screws, pegs, hooks, soldering, bolts and an insert or slide track wherein the rack can be slid into place.

To moisten a wiper in wetting chamber 20, top wall 28 of wetting chamber 20 is lifted open via handle 40 to provide access to rack 56. Wipers are then placed on rack 56 and top wall 28 is lowered to close wetting chamber 20.

Liquid is then pumped into wetting chamber 20 through tubing 54. Upper and lower tubing 42 and 48 carry the liquid to upper and lower spray nozzles 46 and 52, which then spray liquid on any wipers placed on rack 56. Once the target saturation level has been reached, pump 14 halts and liquid stops flowing from tubing 54, 42 and 48 to nozzles 46 and 52.

The configuration of nozzles 46 and 52 provide spray coverage across the entire surface of the wiper. This allows for a uniform saturation level within the wiper itself.

Other embodiments include alternative access means to wetting chamber 20. One such example is where one of the sidewalls 32, 34, 36, or 38 is attached to wetting chamber 20 on one side and can be pulled open to provide access to wetting chamber 20.

Another embodiment includes having the interior wetting mechanism of wetting chamber 20 on a platform and the sidewalls 32, 34, 36, or 38 pull out like a drawer. Therefore, when the access means is open, the device including rack 56 and sprayer nozzles 46 and 52 automatically slide out also.

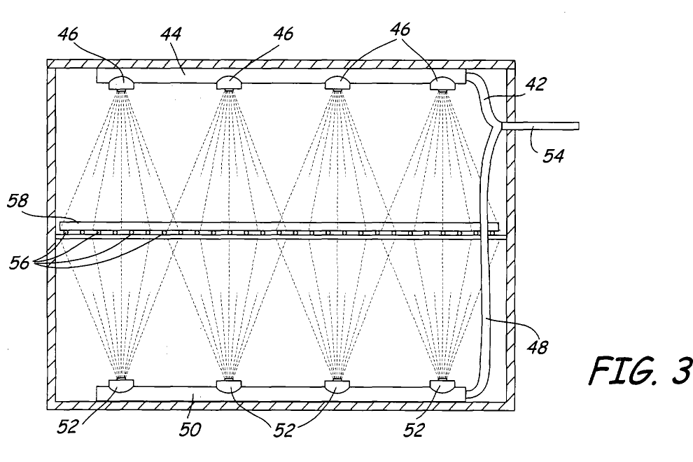

FIG.3 is a diagrammatic view of the interior of wetting chamber 20 in use. The interior of wetting chamber 20 includes upper tubing 42, upper manifold 44, upper set of spray nozzles 46, lower tubing 48, lower manifold 50, lower set of spray nozzles 52, connector 54 and rack 56. Wiper 58 is shown positioned on rack 56.

Upper spray nozzles 46 of wetting chamber 20 are connected to upper manifold 44. Upper spray nozzles 46 are spaced apart from one another to provide greater spraying or misting coverage. An example of a nozzle configuration includes a centrally placed line of four evenly spaced apart nozzles spray 46. Other suitable spray nozzle configurations and number of spray nozzles may also be used. Examples of such include, but are not limited to a set of fine nozzles with one nozzle in each corner section of wetting chamber 20 and one centrally located nozzle or three linear rows of nozzles. Lower spray nozzle 52 are affixed to lower manifold 50. Lower spray nozzles 52 can have the same configuration as upper spray nozzles 46 or a different one—to maximize spraying coverage. Wiper 58 is set on rack 56 so that it is centrally located between upper and lower spray nozzles 46 and 52.

Wiper 58 on rack 56 is moistened when pump 14 is turned on to 20 draw liquid from the liquid supply 12 and deliver the liquid under pressure to wetting chamber 20. The liquid travels through tubing 54, upper and lower tubing 42 and 48 to upper and lower spray nozzles 46 and 52. The liquid is directed to wiper 58 from spray nozzles 46 and 52. Once the feedback device 24 senses that wiper 58 is properly moistened, pump 14 turns off and the liquid flow ceases.

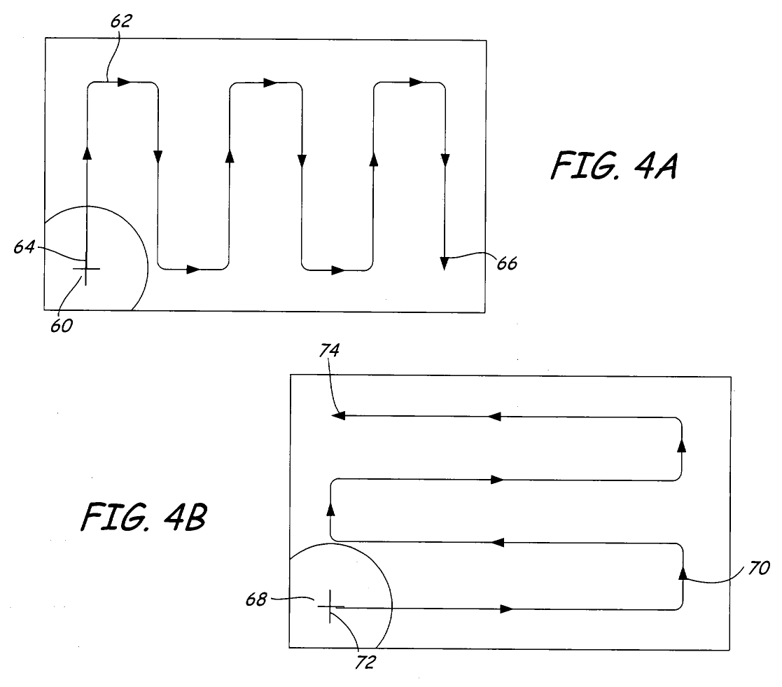

FIGS. 4A and 4B are pattern diagrams of alternative spray nozzle configurations where the nozzles are movable. These configurations require fewer nozzles while still ensuring uniformity of saturation between wipers and within the wipers itself. These configurations allow for a minimum of one spray nozzle to move in a specified pattern while dispensing liquid to the wiper.

In the pattern illustrated in FIG. 4A nozzle 60 moves across the width of wetting chamber 20 in a transverse serpentine pattern along the length of wetting chamber 20. The pattern includes at least one nozzle 60, a path 62, a starting point 64 and an end point 66. Path 62 begins in a corner of wetting chamber 20 at pathway starting point 64. Nozzle 60 begins spraying at starting point 64. From starting point 64, nozzle 60 moves along path 62 to end point 66, at which point the wiper is uniformly saturated. This pattern allows the entire wiper to be sprayed in one pass.

FIG. 4B illustrates an alternative spray nozzle configuration for the movable nozzle. This pattern moves nozzle 68 in a longitudinal serpentine pattern that is in wetting chamber 20. The pattern includes at least one nozzle 68, a path 70, a starting point 72 and an end point 74. Nozzle 68 begins spraying at starting point 72. From starting point 72, nozzle 68 travels path 70 to end point 74. Nozzle 68 continually sprays liquid as it moves along path 70. When nozzle 68 reaches end point 74, the wiper is uniformly saturated. This pattern also allows the wiper to become uniformly saturated with one pass.

The spray pattern for the movable spray nozzles can also include any other pattern that would spray the entire wiper with liquid. These patterns could also include, but are not limited to spraying the perimeters of the wiper from the outside edges inward or advancing the spray nozzles in width wise section. These configurations can be implemented with only one set of nozzles or with both upper and lower spray nozzles 46 and 52.

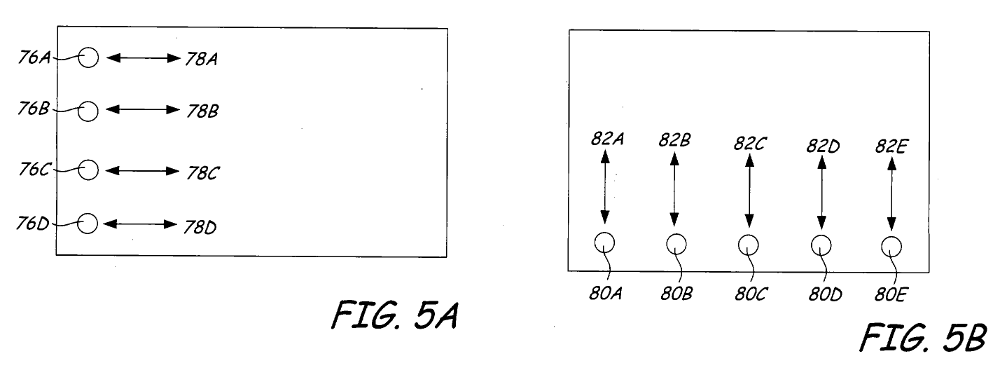

FIGS. 5A and 5B are pattern diagrams of additional alternative spray configurations. In these configurations, a set of nozzles is placed in a linear row in wetting chamber 20. The nozzles then move within wetting chamber 20 to apply liquid to the wiper.

FIG. 5A illustrates a spray pattern configuration for movable nozzles. The pattern includes a set of nozzles 76A-76D and a spray path 78A-78D, respectively, for each of the nozzles. In the illustrated example, the nozzles 76A-76D are placed in a linear row across the width of wetting chamber 20. Set of nozzles can include any number of nozzles appropriate for the size of wetting chamber 20. Each nozzle 76A-76D moves within wetting chamber 20 along its corresponding path 78A-78D. As nozzles 76A-76D move longitudinally across the length of wetting chamber 20 they spray liquid on the wiper. This configuration allows the wiper to become saturated with one pass, or in multiple passes.

This movable spray pattern could also be configured into other patterns such as the pattern illustrated in FIG. 5B where spray nozzles 80A-80E are placed in a linear row across the length of wetting chamber 20. Nozzles 80A-80E move transversely across wetting chamber 20 on paths 82A-82E, respectively. As nozzles 80A-80E move along paths 82A-82E, nozzles 80A-80E continuously spray liquid to provide for uniform saturation the wiper.

These configurations can be implemented with only one set of nozzles or with both upper and lower spray nozzles. Of course, spray nozzle types and positions relative to the wipers must be chosen to ensure uniform application of liquid.

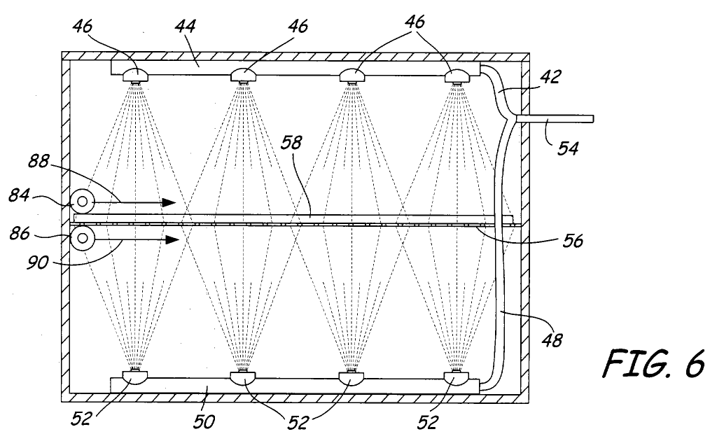

FIG. 6 is a diagrammatic view of a cross section of the interior of an alternative embodiment of the wetting chamber. In this alternative embodiment, wetting chamber 20 includes pressure rollers 84 and 86.

In the embodiment illustrated in FIG. 6, pressure roller 84 is located above rack 56 and wiper 58. A second pressure roller 86 is positioned below the rack 56. In alternative embodiments, a single roller could be used and second pressure roller 86 would be eliminated or more rollers could be added to wetting chamber 20. In this embodiment, the preferred rack 56 is a flat sheet rack with holes for liquid to drain through.

To moisten a wiper to a target saturation level, wiper 58 is placed into wetting chamber 20. A target saturation level is inputted in the user interface 18 (FIG. 1), which correlates with the pounds of force exerted by pressure rollers 84 and 86. Nozzles 46 and 52 then spray wiper 58 until it is completely saturated.

Once wiper 58 is completely saturated, pressure rollers 84 and 86 drive to the other end of wetting chamber 20, with wiper 5 8 in between pressure rollers 84 and 86, to obtain the target saturation level. Pressure rollers 84 and 86 are driven across wetting chamber 20 in the direction of arrows 88 and 90. Once pressure rollers 84 and 86 reach the opposite side of wetting chamber 20, the rollers 84 and 86 will roll back to their original position. Wiper 58 is then ready for use.

Other embodiments of this alternative with pressure rollers could include, but are not limited to, fixed position pressure rollers in conjunction with a moving rack or wiper.

The moistening device of the present invention provides for moistening cleanroom wipers uniformly and repeatably to a desired target saturation level. The invention also provides for saturation of the wiper for the first time at the point of use. This allows a user to pick a saturation level and have the comfort of knowing that each wiper moistened by the device will have that specified saturation level within the entire wiper and across the layers of a small stack of wipers.

Although the present invention has been described with reference to preferred embodiments, workers skilled in the art will recognize that changes may be made in form and detail without departing from the spirit and scope of the invention.

1. A method for controlling a moisture level of a material to be used in a cleanroom, the method comprising;
placing the material in a wetting chamber;
applying a cleaning solution to the material; and
controlling a saturation level of the cleaning solution in the material.

2. The method of claim 1 wherein controlling the saturation level comprises controlling the applying of cleaning solution to the material as a function of electrical conductivity of the material.

3. The method of claim 1 wherein controlling the saturation level comprises controlling the applying of cleaning solution to the material as a function of mass of the material.

4. The method of claim 1 wherein controlling the saturation level comprises controlling the applying of cleaning solution to the material as a function of time.

5. The method of claim 1 wherein controlling saturation level comprises applying pressure to the material to reduce the saturation level of the material to a target saturation level.

6. A device for moistening a material used in a cleanroom to a target saturation level, the device comprising:
a chamber;
a rack positioned in the chamber for holding the material;
a liquid supply of a liquid;
an applicator in the chamber for applying the liquid to the material;
a delivery system for delivering the liquid from the supply to the applicator; and
a control system for controlling the amount of liquid applied to the material based on a parameter related to the target saturation level of the material.

7. The device of claim 6 wherein the control system controls the amount of liquid dispensed to the material as a function of electrical conductivity of the material.

8. The device of claim 6 wherein the control system controls the amount of liquid dispensed to the material as a function in mass of the material.

9. The device of claim 6 wherein the control system controls the amount of liquid dispensed to the material as a function of a time duration.

10. The device of claim 6 wherein the rack is centrally positioned within the chamber.

11. The device of claim 6 wherein the applicator includes a pump for pumping the liquid under pressure and a nozzle for applying the liquid.

12. The device of claim 6 wherein the control system includes a pump control for controlling the amount of liquid applied to the material.

13. The device of claim 6 wherein the control system includes a user interface for providing a user input signal representing the target saturation level.

14. The device of claim 6 wherein the control system includes a feedback sensor for providing a feedback signal representing the parameter.

15. The device of claim 6 wherein the control system includes a shut off sensor for providing a signal to the control system to disengage from the application of liquid to the material when the chamber is in an open position.

16. The device of claim 6 wherein the chamber has a drain for draining excess liquid.

17. The device of claim 16 wherein the drain leads to a liquid collection system.

18. A device for moistening a material used in a cleanroom, the device comprising:
a rack for holding the material;
an applicator spaced from and directed toward the rack for applying a liquid to the material; and
a control system for controlling application of liquid to the material based on a desired saturation level of the material.

19. The device of claim 18 wherein the applicator is movable with respect to the rack.

20. The device of claim 18 wherein the applicator comprises a plurality of nozzles.

21. The device of claim 18 wherein the plurality of nozzles has a first set of nozzles positioned above the rack and a second set of nozzles positioned below the rack.

22. The device of claim 18 wherein the control system controls the amount of liquid dispensed to the material as a function of conductivity of the material.

23. The device of claim 18 wherein the control system controls the amount of liquid dispensed to the material as a function of mass of the material.

24. The device of claim 18 wherein the control system controls the amount of liquid dispensed to the material as a function of time.

25. The device of claim 18 wherein the machine has a drainage and collection system.

26. A device for moistening a material used in a cleanroom to a desired saturation level, the device comprising:
a chamber;
a rack positioned in the chamber for holding the material;
an applicator for applying liquid to the material;
a user interface for providing a user input signal;
a feedback system for providing a feedback signal which is a function of the saturation level of the material; and
a control system for controlling the amount of liquid applied to the material as a function of the user input signal and the feedback signal.

27. The device of claim 26 further comprising a pressure applicator for applying pressure to the material to reduce liquid content of the material.
